(19) United States
(12) Patent Application Publication
(10) Pub. No.: US 2005/0222581 A1
(43) Pub. Date: Oct. 6, 2005

(54) Multiple lumen access sheath

(75) Inventors: Frank J. Fischer JR., Bloomington, IN (US); Walter N. Ryan, Bloomington, IN (US)

Correspondence Address: BRINKS HOFER GILSON & LIONE, P.O. BOX 10395, CHICAGO, IL, 60610, US

(73) Assignees: Vance Products Incorporated, d/b/a; Cook Urological Incorporated

(21) Appl. No.: 11/089,063

(22) Filed: Mar. 24, 2005

Related U.S. Application Data
(60) Provisional application No. 60/557,828, filed on Mar. 30, 2004.

(51) Int. Cl. A61F 11/00

(52) U.S. Cl. 606/108; 600/121

(57) ABSTRACT

A multiple-lumen access sheath for use with an endoscopic or laparoscopic device. The sheath includes a flared funnel on a proximal end of the access sheath for ease of handling by a user. The sheath also includes an elongated portion extending from the funnel to a distal end of the sheath, the elongated portion comprising a first lumen and at least one additional lumen for substantially the length of the elongated portion. There is also an interface for the at least one additional lumen on the proximal end.

This application claims the benefit of the filing date under 35 U.S.C. § 119(e) of Provisional U.S. Patent Application Ser. No. 60/557,828, filed on Mar. 30, 2004, which is hereby incorporated by reference in its entirety.

### FIELD OF THE INVENTION

This patent relates to the field of medical devices, and in particular to devices for the fields of endoscopic and laparoscopic surgery.

### BACKGROUND

Access sheaths, such as ureteral access sheaths, may be used to gain access to body cavities and lumens during endoscopic and laparoscopic surgery, and by other procedures that generally use minimally invasive techniques. Thus, a ureteral access sheath may be used with an endoscope for finding and removing kidney stones, and may also be used in other applications, such as for access to bile ducts. Other applications for which an access sheath has been used include vascular procedures, as well as procedures requiring gastro-intestinal access, uterine access, and bronchial access. Thus, sheaths may be used in combination with endoscopes, hysteroscopes, sigmoidoscopes, bronchoscopes, and many other types of instruments meant for minimally-invasive techniques.

Using a sheath provides for a way to protect the tissues of a patient during a procedure. For instance, if a kidney stone is to be removed, a retrieval basket may require many passages back and forth across a patient's ureter to remove stone fragments. A wire basket or other device is first passed through the ureter to retrieve stone fragments, and then passes back through to remove the captured fragments. Passing the basket across an access sheath instead of the ureter itself avoids trauma to the ureter and surrounding tissues.

One problem that is common to all procedures in which these devices are used is that more and more is expected from the surgeon and operating team. For instance, now that an access sheath may be used for access across a ureter, the surgeon may wish to use the sheath for access not only for an endoscope, but also for multiple endoscopic instruments, such as a retrieval basket, a stone “blocker” or backstop, a fiberoptic laser to break up stones, a safety wire, an operating wire, or to provide irrigation or to instill contrast agents. While all these systems are desirable, it is difficult to operate them all at the same time and through the same access sheath.

What is needed is an access sheath that allows the surgeon to best use valuable space in the sheath and in the endoscope or other minimally-invasive device used on the patient. What is needed is a way to better use the space available with an access sheath.

### BRIEF SUMMARY

The problems mentioned above are alleviated with an improved access sheath. The improved access sheath has more than one lumen for additional functions during a medical procedure. One aspect of the present invention is a multiple-lumen access sheath for use with an endoscopic or laparoscopic device. The sheath comprises a flared funnel on a proximal end of the access sheath, an elongated portion extending from the funnel to a distal end of the sheath, the elongated portion comprising a first lumen and at least one additional lumen for substantially the length of the elongated portion, and an integrally-attached interface for the at least one additional lumen on the proximal end.

Another aspect of the invention is a multiple-lumen access sheath. The multiple-lumen access sheath comprises a funnel having an elongated side and a short side on a proximal end of the access sheath, an elongated portion extending from the funnel to a distal end of the sheath, the elongated portion comprising a first lumen having a constant diameter and at least one additional lumen, and an integrally-attached connector for a fluid at a proximal end of the at least one additional lumen.

Another aspect of the invention is a method of making a multiple-lumen access sheath. The method comprises furnishing at least two spring coils and attaching a fluid connector to one of the at least two coils. The method also comprises forming first plastic around the at least two spring coils and assembling the coils, the plastic and the fluid connector with a funnel to form a multiple-lumen access sheath. There are many other embodiments of the invention, a few of which are described in the drawings and discussed below among the preferred embodiments.

### BRIEF DESCRIPTION OF THE DRAWINGS

FIG. 1a is a side view of a first embodiment of a multi-lumen access sheath;

FIG. 1b is a side view of a dilator for use with the first lumen of the multi-lumen access sheath;

FIGS. 1c and 1d are cross-sectional and perspective views of an embodiment of a multi-lumen access sheath;

FIG. 2a is a cross-sectional view of the embodiment of FIG. 1a;

FIG. 2b is a perspective view of the embodiment of FIG. 1a;

FIGS. 3a and 3b are alternate embodiments of a termination of a second lumen in the multi-lumen access sheath; and

FIG. 4 is a flow chart for a process for making a multi-lumen access sheath.

### DETAILED DESCRIPTION OF THE DRAWINGS AND THE PRESENTLY PREFERRED EMBODIMENTS

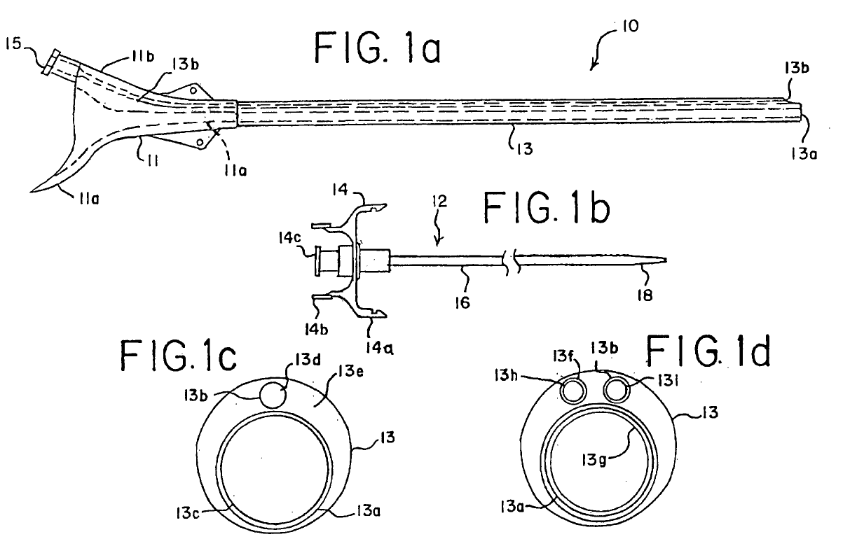

One embodiment of a multi-lumen access sheath has a central, larger lumen for use with an endoscopic device, such as a basket retriever, and a single, smaller lumen which may be used for other purposes. FIG. 1a depicts such an access sheath 10. This multi-lumen access sheath 10 includes funnel 11 and elongated portion 13 which also includes a first larger lumen 13a and a second, smaller lumen 13b. Funnel 11 includes a large flared portion 11a. Funnel 11 also includes generally bowl-shaped portion 11b, which is convex on the outside and concave on the inside. The shape of funnel portions 11a and 11b is adapted to fit the hand of a user. Lumens 13a, 13b extend from the funnel at the proximal end to the distal end of the access sheath, or very close to the distal end. At the proximal end, larger lumen 13a opens into the funnel 11 itself, while smaller lumen 13b terminates in a connector 15. Connector 15 is desirably a Luer lock connector.

The access sheath of FIG. 1a may be used with a dilator 12 depicted in FIG. 1b. Dilator 14 includes a proximal handle portion 14, an elongated portion 16, and a tapered distal portion 18. The elongated portion may be solid or may be hollow. Dilator 12 is flexible, as is the multi-lumen access sheath. Dilator 12 may be used with the access sheath to help dilate a ureter or other body passage. Handle 14 may include locking portions 14a for fitting onto the funnel of an access sheath, and may also include grasping portions 14b for opening locking portions 14a and placing the handle onto the access sheath. Handle portion 14 is desirably injection molded, using medically-acceptable materials such as nylon, acetal, or polyethylene. Elongated portion 16 is desirably extruded or otherwise formed in a separate process. Elongated portion 16 may be assembled onto handle 14 with an adhesive, or they may be snap-fit or otherwise joined together.

One embodiment of a multi-lumen access sheath has a first, large lumen and a single second smaller lumen. Such an embodiment is depicted in FIG. 1c, which depicts a cross-section of a portion of FIG. 1a. Elongated portion 13 includes a first lumen 13a and a smaller second lumen 13b. First lumen 13a desirably is lined on its inner surface with a fluoropolymer liner 13c, such as PTFE or Teflon®. Second lumen 13b is also desirably lined with a fluoropolymer liner 13d on its inner surface. A liner makes the inner surface smooth and lubricious, and eases the passage of devices or fluids through the lumen. The elongated portion itself 13 may be a polymeric material, such as nylon. Any medically acceptable thermoplastic or thermoset material may be used, including PTFE, a fluoropolymer, polyethylene, polypropylene, acetal, urethane, and others. In some embodiments, the second lumen may not have a lining and may instead by defined merely as a lumen of relatively constant diameter that remains when a plug or mandril is removed after the material in the elongated section is formed or consolidated.

Another embodiment of a multi-lumen access sheath is depicted in cross-section in FIG. 1d. In this embodiment, elongated portion 13 includes a first, larger lumen 13a and two smaller lumens 13b and 13f. Each lumen may include an inner liner or coating 13g, 13h, and 13i. The two extra lumens may be used, for example, for inflow and outflow of irrigation fluid.

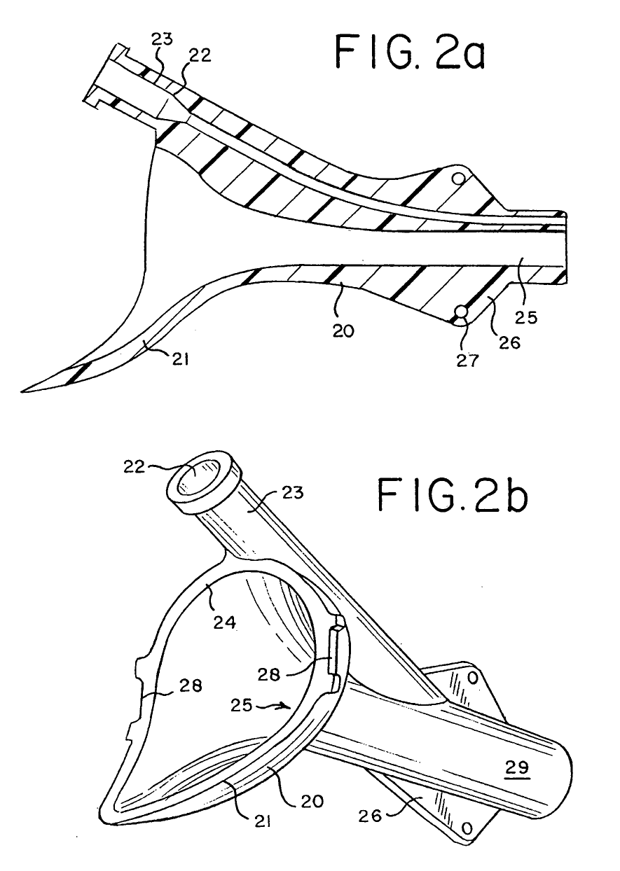

FIGS. 2a and 2b are additional views of the embodiment of FIG. 1a. FIG. 2a is a cross-sectional view of the proximal portion of the embodiment of FIG. 1a. Proximal portion 20 functions as a handle for a user of the device. Proximal portion 20 includes a larger flared portion 21. The outside of flared portion 21 provides additional material for grasping by a user, while the inside helps to guide instruments or materials into lumen 25. Proximal portion 20 also includes one or more ribs 26. The ribs are thin projections from the proximal portion and are meant to help a user orient and grasp the sheath. For instance, a user may place the ribs between joints of a user's adjacent fingers to grasp and steady the access sheath. The ribs may also have a bore 27. Second lumen 22 terminates in a connector 23 at the proximal end of the access sheath. Connector 23 is preferably integrally attached, e.g., by molding the connector to the access sheath, by insert molding a connector to the access sheath, or by adhering the connector with an adhesive.

FIG. 2b is a perspective view of the embodiment of FIG. 2a. Proximal portion 20 includes a wider flared portion 21 for ease of grasping by a user. The inner portion of the wide flared portion also helps to guide instruments or a dilator through the funnel to lumen 25. Proximal portion 20 also includes a narrower, bowl-shaped portion 24 opposite the wide flared portion. Connector 23 with lumen 22 is preferably adjacent this portion.

Between wide and narrow portions 21, 24 are two attachment portions 28. The attachment portions are designed for attachment of a dilator 12 or other object normally used with the access sheath. The proximal portion ends with a round or elliptical elongated portion 29 and two ribs 26. The ribs are typically located at 180° on the elongated portion for orientation to a user's hand.

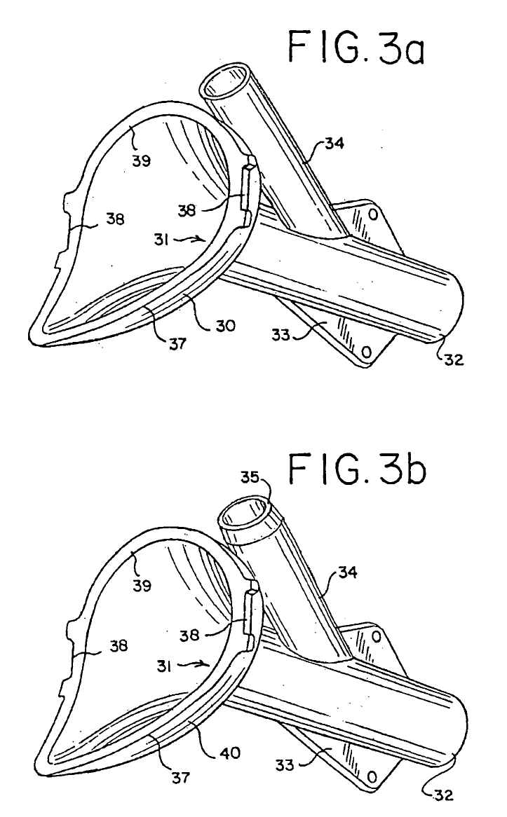

As mentioned above, one embodiment of the invention includes a fluid connector, such as a Luer lock connector, for one of the lumens in the sheath. Other embodiments may use different terminations for one of the lumens. FIGS. 3a and 3b depict two such additional embodiments. In FIG. 3a, multi-lumen access sheath handle 30 includes a first lumen 31, an elongated portion 32 and ribs 33 as shown. An additional lumen, not shown, terminates in a protrusion or interface 34, so that users have access to the lumen. In FIG. 3b, the sheath 40 has the additional lumen terminate in an interface with a beveled or chamfered surface 35, so that a user may more easily introduce a guide wire or other medical device into the additional lumen. Access sheaths 30, 40 have a longer proximal portion 37, a shorter, bowl-shaped proximal portion 39, and interfaces 38 for attaching a dilator.

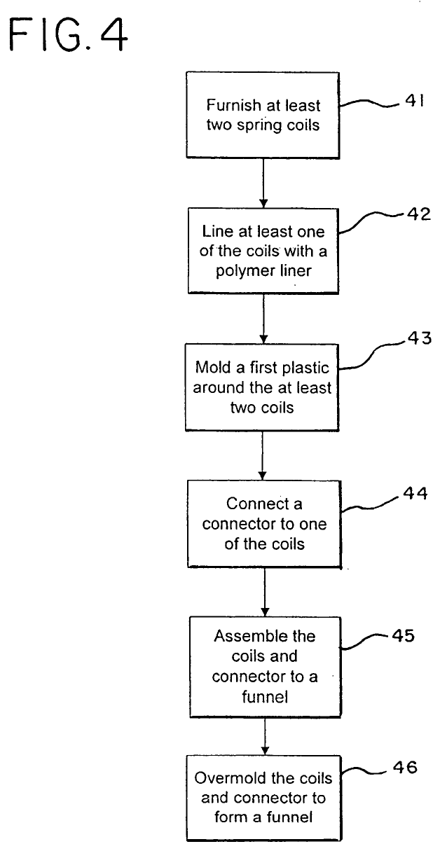

In one embodiment of the invention, a multi-lumen access sheath is made by assembling and molding a number of parts. FIG. 4 depicts a process for making a multi-lumen access sheath. The process includes a step of obtaining stainless steel and forming 41 the stainless steel into at least two coil springs. A coil spring and a coating on the inner diameter, if any, define a lumen for the sheath. Alternatively, one may purchase coils from a vendor that makes stainless wire into small diameter coils. One such vendor is Star Guide, Denver, Colo., USA.

One of the coil springs is desirably larger than the other(s), for forming a large lumen in the access sheath. One or more of the coils is desirably coated 42 with a polymer liner to make the inner diameter of the coil smooth and relatively friction-free. Vendors may also furnish the coils coated with the desired material. The coils are assembled 43 together by molding a plastic around them. A connector is connected 44 to one of the coils. The step of connecting may occur before the step of assembling the coils together.

The coils and connector are then assembled 45 to a funnel. In one embodiment, the coils and connector may be assembled to a separately-molded funnel by using an adhesive or other connection. In another embodiment, the funnel may be overmolded 46 onto the assembled coils and connector, as by insert molding. The insert molding may be accomplished by an injection molding process, a transfer molding process, or a compression molding process. Other molding processes may also be used to connect the coils and connector to a funnel or proximal portion of the access sheath.

Applications of a Multiple-Lumen Access Sheath

There are many possible applications for a multiple-lumen access sheath. One possibility is in the removal of kidney stones from a urinary tract of a patient. In one situation, a patient may have a large kidney stone in a kidney which will not pass through a ureter. A multiple-access sheath with a first lumen of 9.5 Fr, 10, Fr, 12 Fr, or 14 Fr may be used in combination with an endoscope. This sheath may have a single additional lumen with a diameter of about 3 Fr (about 0.040 inches). The sheath may be placed into the urinary tract by first inserting a guide wire and using a smaller lumen of the multiple-lumen access sheath to follow the guide wire into the ureter and kidney of the patient.

The guide wire may then be removed. An endoscope with optics and a working channel is then inserted using the first lumen of the sheath. The surgeon may locate the kidney stone with the optics portion of the endoscope, and may then wish to use a fiberoptic laser in the working channel of the endoscope. There may be sufficient room in the working channel of the endoscope for a fiberoptic laser to fragment the stone, and there may also be room for a retrieval basket to retrieve the fragments. One such retrieval basket is a 4-wire NCircle® retrieval basket with a 2.4 Fr PTFE coated polyimide sheath, sold by Cook Urological Inc., Spencer, Ind. The additional lumen in the access sheath may then be used to irrigate the field and clear the viewing lens of the endoscope.

In other embodiments, the additional lumen in the access sheath may be used for the fiberoptic laser, while the working channel of the endoscope may be used for an irrigation channel and the retrieval basket. In another embodiment, the additional lumen may be used for a retrieval basket, while the working channel in the endoscope may be used for the fiberoptic laser and for an irrigation system. The additional lumen gives an additional degree of freedom to the surgeon, who may now choose which lumen for which instrument. There may also be an advantage in simply the spacing or location of the lumens in the access sheath as placed in the patient that makes the additional lumen or lumens desirable for the surgeon. In embodiments with two additional lumens, one may be used as a source of irrigation fluid from one connector, while the other may be connected to a source of vacuum, and used to evacuate irrigation fluid from the operating field.

Embodiments of the invention are not limited to ureteral procedures. Similar procedures may be performed for exploration of the common bile duct. A multi-lumen access sheath may be introduced into the abdominal cavity and if necessary, dilation may take place with a balloon angioplasty catheter. A 5.0 Fr Accent® balloon angioplasty catheter with an 8 mm balloon, available from Cook Incorporated, Bloomington, Ind., may be inserted into the principal channel of the access sheath. The angioplasty catheter may be used to dilate the cystic or common duct, or both. An NCircle® retrieval basket may be inserted through the additional lumen in the access sheath in order to retrieve bile stones if they are sufficiently small. If the bile stones must be fragmented, the additional lumen may be used sequentially for a fiberoptic laser and then for a retrieval basket to gather the stone fragments. The additional lumen saves time for this procedure and allows the surgeon another degree of freedom in the operating room. This is accomplished with minimal trauma to the patient's tissues.

Another application for a multi-lumen access sheath is for vascular access. One possible application may be for contralateral access to the iliac artery after an initial wire guide position is established. A smaller lumen of the multi-lumen access sheath may be inserted, following the wire guide. This allows direct positioning of the sheath and a dilator, if desired. Once in position, the dilator may be used for vascular access. A Luer lock connector for the additional lumen may be connected to a valve and used for additional vascular access. For instance, instead of depending only on a single lumen for dilation and access, the valve and connector may be used for sampling, aspiration, or for the delivery of medications or marking fluids.

Another application is gastrointestinal access. A multiple-lumen access sheath may be placed as part of a catheter system for access to the stomach and intestinal tract of a patient. The access sheath may be placed using by first using a wire guide and inserting the access sheath using one of the lumens of the access sheath. The wire guide may be withdrawn, if desired, and the wire guide lumen may then be used, for instance, for aspiration of gastric contents in the stomach while a larger lumen is used for feeding of the jejunum. Other uses include direct percutaneous nephrostomy access, in which the second lumen may be used to secure a safety wire, or to place an occlusion device to prevent stone fragments from migrating down the ureter.

Forming Lumens from Coils

The sheath desirably includes spring coils to form the lumens. Coils are formed by taking thin metal wire, preferably stainless steel, and wrapping the wire into a coil. Round wire or flat wire may be used. In one embodiment, 304 stainless steel flat wire measuring 0.004 inches (0.10 mm)×0.012 (0.30 mm) inches is wrapped into a tight coil having an outer diameter of about 0.164 inches (4.17 mm) and an inner diameter of about 0.156 inches (3.96 mm). A polymer coating, such as a coating or tube made from Teflon®, PTFE, or other fluoropolymer, is applied to the inner diameter. The coating may be applied at any convenient time during the process. Other coatings may be used.

One or more additional lumens may be formed in the sheath by also using metal wire to form a smaller wire coil. For instance, round stainless steel wire with a diameter of 0.00075 inches (0.02 mm) may be used to wrap a coil with an outer diameter of 0.040 inches (1.02 mm) and an inner diameter of about 0.0385 inches (0.98 mm). In addition to stainless steel, other medically acceptable materials may also be used, such as nitinol, tungsten, platinum, MP35N, or combinations of metals, such as clad alloys. One example of a clad alloy is MP35N/silver.

Of course, the lumen need not be lined, but may instead be formed by using a removable mandrel to “reserve space” in the sheath during formation of the elongated portion, and then removing the mandril at some point after formation of the elongated portion. Thus, a mandril made from PTFE, Teflon®, or other slick, non-binding material may be used. Alternately, or in addition, an antiseize compound may be used to coat a mandril. The compound will preferably not volatilize during molding and should not prevent curing consolidation of the thermoplastic or thermoset material that forms a considerable part of the elongated portion.

Assembling the Coils into a Sheath

Once the coils are formed, and desirably coated, they are assembled into a sheath. This assembly process desirably takes place in at least two steps. There is typically, but not necessarily, a larger coil and a smaller coil. The larger coil will be used for the desired access, such as vascular access or access to a ureter. The smaller coil(s) may be used for one or more ancillary tasks, such as irrigation, removal of irrigation or saline, guide wire insertion, and the like. The smaller coil may require a connector in order to function effectively. Thus, if irrigation fluid is to be introduced through an auxiliary lumen, a connector to a source of irrigation fluid is needed. If evacuation of fluid or irrigation is desired, a connector suitable for a vacuum connection may be desirable. Even if the additional lumen is used only for a guide wire, it will be necessary to terminate the auxiliary lumen in such a manner that operating room personnel have access to the lumen. Thus, the proximal portion of the sheath, the handle, desirably has an interface or termination point for the additional lumen that may be viewed and accessed by operating room personnel.

The above description is not meant to be limiting, and only a few of the possible embodiments have been described. For instance, emphasis has been placed on flexible multiple-access sheaths. However, there are also applications for sheaths with more rigidity; these sheaths may be made with lumens defined by tubing rather than coiled springs. The tubing may be rendered flexible by a series of cuts, such as spiral cuts in one or more regions of the access sheath for flexibility in selective regions. The tubing may be cut or slit, for example, by laser cutting, as described in co-pending application 10/617,580, now U.S. Pat. No. ______, assigned to the assignee of the present application, and which is hereby incorporated by reference in its entirety.

The coils or tubing that define lumens in the multiple-lumen access sheath are preferably lined with a smooth polymer, in order to minimize friction as objects, such as retrieval baskets, wire guides, or endoscopes are passed through the lumens. While fluoropolymers as described above make excellent liners, other materials may also be used, such as urethanes and olefins. The liners themselves may also be coated, if the application requires, with lubricious or hydrophilic coatings, although these coatings are typically activated by moisture and may not be necessary for most applications.

An interface or connector will be very helpful in order for operating room personnel to efficiently use the at least one additional lumen in the access sheath. Even if the interface is only a chamfered or beveled surface on the top of the proximal portion, it will help operating room personnel use the additional lumen. The lumen is preferably a connector, such as a Luer lock, or other “universal” connector that is easily usable to connect to syringes, sources of vacuum, fluid plumbing devices such as valves, or other medical devices. While a connector is preferred, any interface that assists operating room personnel in using the one or more additional lumens is intended.

The elongated portion of the multiple-lumen access sheath is actually a relative small fraction of the material used in the sheath, because much of the volume is taken up by the tubing or coils that define the lumens. The elongated portion may be made from one or more layers of heat-shrink tubing, such as urethane or PTFE heat-shrink tubing, wrapped around the coils or tubing. Other materials, such as nylons or olefins, may also be used so long as they are medically acceptable. Films or thin strips of material may also be wrapped or formed around the tubing or coils to form the elongated portion, rather than merely shrinking heat-shrink tubing. The assembled coils or tubes and polymeric materials may then be placed into a mold or form and the polymeric material consolidated and molded. This is another way to integrally-connect a connector.

The multiple-access sheath is desirably assembled in stages, as described above, or it may be molded at once. For instance, coils and liners defining the lumens may be prepared and wrapped with polymeric materials for consolidation into an integrated elongated portion. A connector or interface is desirably added to at least one coil defining a lumen, preferably a smaller coil, at this stage. The connector or interface may be adhesively bonded to the coil, or may be mechanically connected or fitted to the coil. A subassembly of the elongated portion and the connector may then be insert molded to a proximal portion. This may be accomplished by any number of molding techniques.

Alternatively, the entire sheath may be insert molded in a single step by placing spring coils or tubing into a mold, and then injecting plastic or compressing or transferring a polymeric material to define the access sheath and its proximal portion, including an interface or connector for the additional lumen. A single-step method may not be desirable because of the elongated nature of the sheath and the expense of a tool large enough to accommodate what may be a very long sheath, up to 35 or 40 cm long. Also adding to tooling expense is the complexity of a proximal portion having both a funnel portion and an additional connector. The multiple-access sheath may be made by this method. However, a two-step process as described above, will be more effective in controlling the process and the cost, and will likely result in a more uniform product.

It is intended that the foregoing detailed description be regarded as illustrative rather than limiting, and that it be understood that it is the following claims, including all equivalents, that are intended to define the spirit and scope of this invention.

1. A multiple-lumen access sheath for use with an endoscopic or laparoscopic device, the sheath comprising:
a flared funnel on a proximal end of the access sheath;
an elongated portion extending from the funnel to a distal end of the sheath, the elongated portion comprising a first lumen and at least one additional lumen for substantially the length of the elongated portion; and
an integrally-attached interface for the at least one additional lumen on the proximal end.

2. The sheath of claim 1, wherein the elongated portion comprises plastic and the first lumen is defined by a wound spring coil having an inner liner.

3. The sheath of claim 1, wherein the first lumen of the elongated portion is defined by a stainless steel wound spring coil with a fluoropolymer liner.

4. The sheath of claim 1, wherein the at least one additional lumen is defined by a stainless steel wound coil spring with a fluoropolymer liner.

5. The sheath of claim 1, wherein the proximal end further comprises two ribs at 180° on an outside of the sheath.

6. The sheath of claim 1, wherein the sheath comprises polymeric material.

7. The sheath of claim 1, wherein the at least one additional lumen comprises a first additional lumen connected to the interface, and a second additional lumen.

8. The sheath of claim 1, further comprising a dilator adapted to fit onto the funnel of the sheath.

9. The sheath of claim 1, wherein the integrally-attached interface comprises a connector for a fluid.

10. A multiple-lumen access sheath, comprising:
a funnel having an elongated side and a short side on a proximal end of the access sheath;
an elongated portion extending from the funnel to a distal end of the sheath, the elongated portion comprising a first lumen having a constant diameter and at least one additional lumen; and
an integrally-attached connector for a fluid at a proximal end of the at least one additional lumen.

11. The sheath of claim 10, wherein the first lumen and the at least one additional lumen are formed by tubing or spring coils with polymer liners.

12. The sheath of claim 10, wherein the connector is a Luer lock connector.

13. The sheath of claim 10, wherein the funnel is adapted to fit the hand of a user.

14. The sheath of claim 10, further comprising a dilator.

15. A method of making a multiple-lumen access sheath, the method comprising:
furnishing at least two spring coils;
attaching a fluid connector to one of the at least two spring coils;
forming a first plastic around the at least two spring coils; and
assembling the molded plastic and the fluid connector to a funnel to form a multiple-lumen access sheath.

16. The method of claim 15, wherein the step of assembling is accomplished by placing the molded plastic and connector into a mold and overmolding with a plastic different from the first plastic.

17. The method of claim 15, wherein the step of assembling is accomplished using an adhesive.

18. The method of claim 15, further comprising assembling a dilator with a handle into the sheath.

19. The method of claim 15, wherein the step of molding a first plastic is accomplished by heating and shrinking heat-shrink tubing wrapped around the at least two coils.

20. The method of claim 15, wherein the spring coils are furnished with a polymeric lining.
